(19) United States
(12) Patent Application Publication
(10) Pub. No.: US 2005/0064567 A1
(43) Pub. Date: Mar. 24, 2005

(54) Anaerobic hydrogen-producing process

(76) Inventors: Jiunn-Jyi Lay, Kaohsiung (TW); James I. Chang, Kaohsiung (TW); Kuo-Shu Fan, Kaohsiung (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/667,430

(22) Filed: Sep. 23, 2003

(51) Int. Cl. C12P 3/00; C12P 1/04

(52) U.S. Cl. 435/168; 435/170

(57) ABSTRACT

Anaerobic hydrogen-producing process includes steps of producing hydrogen from organic waste under anaerobic condition, utilizing microorganisms, and of purifying hydrogen directly useable as fuel, and then transformed to become electric energy by a fuel cell.

### BACKGROUND OF THE INVENTION

1. Field of the Invention

This invention relates to processes or an anaerobic hydrogen-producing process, particularly to one including steps of fermenting organic waste under anaerobic condition for producing hydrogen, which can be transformed into energy by a fuel cell for directly generating electricity. As hydrogen is cleaner than methane obtained through processes of fermenting under a traditional method under anaerobic condition, and in addition, the temperature of the gas to be produced can be lowered, and waste treatment and energy production can be carried out at the same time,

2. Description of the Prior Art

Hydrogen reacts with oxygen to offer not only heat energy but also harmless and odorless water steam, and can be used as fuel, far cleaner than fossil fuel. However, hydrogen does not singly exist on the earth, has to be obtained by transitional techniques of electrolysis of water and thermolysis of fossil fuel. But it requires a large amount of energy in its production, resulting in high cost, impossible to take place of the fossil fuel to become a major fuel so far.

In recent years, a device to transform hydrogen into energy, called ‘a fuel cell”, has been successfully developed, and energy loss in transportation of liquefied hydrogen is far less than that of electric energy, so utilization and development of hydrogen has been gradually made much of. According to the estimation of American Energy Department, hydrogen may march into commercialized energy market about 2020, and it can be transformed from fossil fuel for a short term. But the deposit of fossil fuel is limited, and will be depleted someday in the future. So from a long perspective point of view, hydrogen will become a main source of energy, gotten from recyclable matters.

In the end of the 19th century, scientists have already found out that hydrogen can be obtained from algae and bacteria, and large number of microorganisms possible to produce hydrogen have been discovered these decades. It is generally deemed by scientists that hydrogen production has direct relation with the total metabolism of microorganisms, and electron released out by microorganisms will combine with hydrogen ions through catalyzing of hydrogenase to become hydrogen molecules. So hydrogen-producing process is actually a process of disposing electrons generated during metabolism and scholars call it “Hydrogen Valve” moderating energy metabolism, compared to production of hydrogen

Before 1990, most of the research works in the area of microbial hydrogen production are restricted in the fundamental and academic studies, such as the characterization and separation of bacterial species, reaction mechanism, etc. In 1970, US National Science Foundation began to fund research projects in this area and held symposiums to disseminate the findings. Though most of the research projects are focused on the hydrogen production through photosynthesis rather than anaerobic process, this research field has become one of major fields of research in microbiology. For more than two decades, research and results of hydrogen production through microbiology have been disclosed in microbiological periodicals, with remarks and retrospective treatises specific to this field published every several years between. The fundamental mechanism and theory for the microbial production of hydrogen have been well established and generally accepted since then.

There are many kinds of hydrogen-producing bacteria, but photosynthetic bacteria and anaerobic shuttle-shaped spore bacteria have the largest potential. Ministry of International Trade and Industry of Japan began to promote “New Sunlight Project” since 1993, wherein the research project of hydrogen production through microbes concretely includes a system of photosynthetic hydrogen production, promotion of hydrogen enzyme possible to transform light into energy so as to enhance hydrogen production, sift and analysis of bacteria, separation and purification of hydrogen, utilization and development of remaining biological body and its spin-off, integration of the system, using solar energy and hydrogen production from waste organic water, etc.

In 1990, US Congress passed a law “Hydrogen Research, Development and Demonstration Program Act”, and the Energy Department began to carry out hydrogen production and research and development. “Energy Policy Act” passed in 1992 concretely demanded the government to make estimation and development of producing hydrogen from recyclable resource, and the Recycled Energy Research Institute had to carry it out by producing hydrogen by means of reaction of bacteria with photosynthesis as a main means.

The research of photosynthetic production of hydrogen in US and Japan are restricted to medium and small reaction tanks, impossible to be enlarged, and far from being commercialized at present owing to the design problem regarding light shining and the reaction tank.

As the anaerobic fermentation has been matured, possible to develop anaerobic hydrogen production technique, with its producing processes easily enlarged, and capable to achieve maximum results with little effort. In the traditional fermentation process, though including a great amount of bacteria for producing hydrogen, also comprises a large amount of hydrogen-utilizing bacteria so that only a little hydrogen is contained in biogas produced. Therefore, activeness of the hydrogen-utilizing bacteria has to be contained so as to boost up production of hydrogen.

At present, an anaerobic treating process of organic waste masses can recycle a large amount of methane from them, not only perfecting its technique but also realizing its commercialization. If proper design of the environmental and operational moderation is mapped out in the methane fermentation process for containing hydrogen-utilizing bacteria and then let organic waste masses produce hydrogen, and then the step of methane fermentation, the original purpose of producing both methane and hydrogen and treating waste can be attained at the same time.

### SUMMARY OF THE INVENTION

The main objective of the invention is to offer a process of producing hydrogen by transforming hydrocarbotate and protein in waste mass coming from agriculture and industry into useful hydrogen, by anaerobic shuttle-shaped spore bacteria present in nature, combining together waste treatment and energy production, based on existing fundamental academic researches.

The anaerobic hydrogen-producing process in the present invention can be applied to waste coming from agriculture and food processing industry and residues containing high carbohydrate and protein, such as wheat lees, yeast solution, dry yeast, sugar cane dregs, animal and plant dregs, etc.

### BRIEF DESCRIPTION OF A DRAWING

This invention will be better understood by referring to the accompanying drawings, wherein:

FIG. 1 is a flow chart of an anaerobic hydrogen-producing process in the present invention.

### DETAILED DESCRIPTION OF THE PREFERRED EMBODIMENT

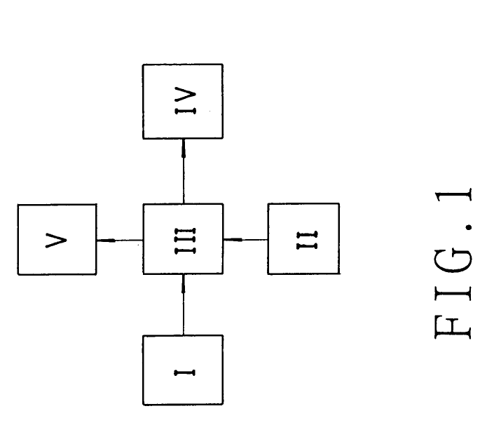

A preferred embodiment of an anaerobic hydrogen-producing process in the present invention includes the following steps, as shown in FIG. 1.

I. A first step of shattering waste into particles of less than 1 mm long and wide, and mixing the particles in water in a proper ratio.

Shattering has an object to break waste organization or to reduce its dimensions so as to facilitate it for transportation and digestion, and equipment used is different depending on the kinds and feature of waste, and shattered waste is mixed with water to have a proper density.

II. A second step of preliminary treatment and concoction of seeding material.

The object of this step is to stimulate germination of anaerobic hydrogen-producing bacteria and to contain activeness of hydrogen-utilizing bacteria; shuttle-shaped spore bacteria has the largest potential, having spores, enduring high temperature more than other bacteria existing mostly in common compost, especially in the bottom layer of manure composting piles. Therefore, compost can be used for seeding material for stimulating germination of bacteria in spore condition, activating its function of hydrogen-producing, removing hydrogen-consuming bacteria in compost, heating and acid sifting should be adopted as preliminary treatments. The treating steps are:

(1) Weed compost or cattle manure compost is placed in a fermentor for three hours, with its temperature adjusted at 80-90 degrees, and then ground into very tiny particles.

(2) The weed compost or cattle waste compost finished the (1) step is mixed with reverse osmosis water, with the ratio (weight ratio), 0.5-1.5/0.5-1.5/10.

(3) After the mixed solution finished the (2) step are stirred and settled, an upper portion of the settled solution is taken out as a seeding solution.

III. A third step of anaerobic fermentation for producing hydrogen.

The main object of this step is to produce hydrogen under anaerobic condition, after waste and the seeding solution are mixed in the fermentation reacting tank in a proper ratio. The reacting steps are as follows.

(1) Place the organic waste (or weeds or compost) (in dry condition), the seeding material, a nutrient in the ratio 1/12.5/0.4-0.5 respectively in a thermostatic reactor; the nutrient has the ingredients as follows:
(a) Ammonium acid carbonate (NH₄HCO₃) 500-600 mg/L
(b) Potassium dihydrophosphate (KH₂PO₄) 35-45 mg/L
(c) Magnesium sulfate MgSO₄.7H₂O) 3-5 mg/L
(d) Sodium carbonate (Na₂CO₃) 0.3-0.5 mg/L
(e) Sodium molybdate (NaMoO₄. 2H₂O) 0.3-0.5 mg/L
(f) Calcium chloride (CaCl₂.2H₂O) 0.3-0.5 mg/L
(g) Manganese sulfate (MnSO₄.7H₂O) 0.5-0.7 mg/L
(h) Ferrous chloride (FeCl₂) 0.10-0.15 mg/L

(2) Add pure water in the basic materials gotten in the (1) step and adjust the density of the basic materials (i.e. waste, seeding material and nutrient together) got in the (1) treatment to 2%-5%.

(3) Adjust the temperature of the reactor to between 35-34 degrees.

(4) Expose a mixed gas of carbon dioxide and nitrogen on the inner surface of the solution in the reactor, with the pressure ratio (P_{co2}/P_{N2}) of the carbon dioxide to the nitrogen being set 3/7.

(5) After sealing the cap of the reactor, disconnect the mixed gas-exposing device for preventing air from entering in the reactor.

The most favorable reacting conditions are:
1. Iron ion is 100-150 mg/L.
2. Ammonium ion is 500-600 mg/L.
3. Phosphate ion density is 1400-1800 mg/L.
4. At first the pH value is 6.0-6.5.
5. The reactor is horizontal, and the rotating speed along the horizontal axis is set 25-35 rpm/m.
6. The temperature of the reactor is controlled at 35-45 degrees.

The condition of the reactor at the beginning is:
1. The basic material (waste) in the continual reactor has its solid density being 2%-5%.
2. The volume ratio of the basic material and the seeding material is 1/3-1/5.
3. The volume ratio of the nutrient and the basic material is 1/15-1/20.
4. The rotating speed of the reactor for stirring is 30-100 rpm/m.
5. The temperature of the reactor is controlled at 36-45 degrees.

After finishing the above steps, hydrogen begins to produce in 1-3 days, and stops in a week or so.

IV. A fourth step of anaerobic fermentation for producing methane gas.

The basic material finishing producing hydrogen in fermentation will produce methane under anaerobic condition by means of methane fermentation in nature.

V. A fifth step of purification of the gas exhausted by hydrogen fermentation.

This step is to remove carbon dioxide mixed in the gas exhausted by the hydrogen producing fermentation to increase the density of hydrogen.

In the above steps I to V, II (preliminary treatment and concocting) and III (anaerobic fermentation for producing hydrogen) are the important improvements made in the invention, and the other steps I (shattering waste and mixing water), IV (anaerobic fermentation for producing methane) and V (purification of the gas exhausted by the hydrogen fermentation tank) are traditional industrial treating ones, applicable to any processes, any equipment, or any method.

While the preferred embodiment of the invention has been described above, it will be recognized and understood that various modifications may be made therein and the appended claims are intended to cover all such modifications that may fall within the spirit and scope of the invention.

1. A anaerobic hydrogen producing process including steps of:
I. A first step of shattering waste into particles of the length and width of less than 1 millimeter and then mixing them in water:
II. A second step of preliminary treating and concocting seeding material:
III. A third step of anaerobic fermenting for producing hydrogen:
IV. A fourth step of anaerobic fermenting for producing methane: and,
V. A fifth step of purifying gas exhausted by hydrogen producing fermentation.

2. The anaerobic hydrogen-producing process as claimed in claim 1, wherein said second (II) step of preliminary and concocting seeding material is to stimulate germination of anaerobic hydrogen-producing bacteria and to contain activeness of hydrogen-utilizing bacteria, said second (II) step comprising treatments of:
(1) Placing weed compost or cattle dung compost in a fermentor for three hours, with the temperature in the fermentor adjusted at 80-90 degrees;
(2) Mixing said seed compost or said cattle dung compost in reverse osmosis water with the ratio (weight ratio) being 0.5-1.5/0.5 1.5/10: and,
(3) Stirring said mixed solution finishing the (2) step and let it settle down and take the upper layer of the solution as a seeding material.

3. The anaerobic hydrogen-producing process as claimed in claim 1, wherein said third (III) step of anaerobic fermenting for producing hydrogen includes batch reacting treatment, said batch reacting treatment comprising:
(1) Placing said organic waste (in dry condition), said seeding material and said nutrient respectively in a thermostatic batch reactor, with their ratio being 1/12.5/0.4-0.5.
(2) Adding pure water in said materials in the (1) treatment, and adjust the density of the basic material to 2%-5%.
(3) Adjusting the temperature of the reactor at 35 to 45 degrees.
(4) Exposing mixed gas of carbon dioxide and nitrogen on an inner surface of said liquid in said reactor, with the pressure ratio (P_{CO2}/P_{N2}) of said carbon dioxide and said nitrogen being 3/7; and,
(5) After sealing the cap of the reactor, disconnect the gas exposing device to prevent air from flowing therein:
Hydrogen beginning to produce in one to three days and stopping in a week or so.

4. The anaerobic hydrogen-producing process as claimed in claim 3, wherein said nutrient materials used in said third (III) step of the batch reacting treatment for producing hydrogen by anaerobic fermentation comprising:
(1) 500 to 600 mg/L of ammonium acid carbonate (NH₄HCO₃);
(2) 35 to 45 mg/L of potassium dihydrophosphate (KH₂PO₄):
(3) 3 to 5 mg/L of magnesium sulfate (MgSO₄.7H₂O):
(4) 0.3 to 0.5 mg/L of sodium chloride (NaCl);
(5) 0.3 to 0.5 mg/L of sodium molybdate (NaMoO₄.2H₂O);
(6) 0.3 to 0.5 mg/L of calcium chloride (CaCl₂.2H₂O);
(7) 0.5 to 0.7 mg/L of manganese sulfate (MnSO₄.7H₂O); and,
(8) 0.10 to 0.15 mg/L of ferrous chloride (FeCl₂):

5. The method for producing hydrogen under anaerobic condition as claimed in claim 1, wherein said third (III) step of anaerobic fermentation for producing hydrogen uses a batch reactor for carrying out batch reaction, said batch reaction needing the most favorable conditions of:
(a) Iron ion density being 100 to 150 mg/L;
(b) Ammonium ion density being 500 to 600 mg/L;
(c) Phosphate ion density being 1400 to 1800 mg/L;
(d) The pH value at the beginning being 6.0-6.5;
(e) Said reactor being horizontal, and having rotating speed along the horizontal axis being 25-35 rpm/m; and,
(f) The temperature of said reactor controlled to be at 35 to 45 degrees.

6. The anaerobic hydrogen-producing process as claimed in claim 3, wherein the continual reactor has initial conditions, said initial conditions comprising:
(1) The solid density of said basic material in said continual reactor being 2% to 5%;
(2) The volume ratio of said basic material and said seeding material being 1/3-1/5;
(3) The volume ratio of said nutrient and said basic material being 1/15-1/20;
(4) The rotating speed of said reactor being 30-100 rpm/m; and,
(5) The temperature of said reactor controlled to be at 35 to 45 degrees.

7. The anaerobic hydrogen-producing process claimed in claim 6, wherein said basic material in said reactor is a substance generating energy such as weeds, compost or the like.

8. The anaerobic hydrogen-producing process as claimed in claim 6, wherein said basic material in said reactor is waste.
